(19) United States
(12) Patent Application Publication
(10) Pub. No.: US 2003/0051261 A1
(43) Pub. Date: Mar. 13, 2003

(54) Plant internal ribosome entry segment

(76) Inventors: Rudy Vanderhaeghen, Merelbeke (BE); Maria Van Lijsebettens, Merelbeke (BE)

Correspondence Address: TRASK BRITT, P.O. BOX 2550, SALT LAKE CITY, UT, 84110, US

(21) Appl. No.: 10/216,540

(22) Filed: Aug. 9, 2002

Related U.S. Application Data
(63) Continuation of application No. PCT/EP01/01026, filed on Feb. 1, 2001.

(30) Foreign Application Priority Data
Feb. 10, 2000 (EP) 00200442.2

(51) Int. Cl. A01K 67/00; A01H 1/00; C12N 5/06; C12N 5/04

(52) U.S. Cl. 800/8; 800/289; 435/325; 435/419; 435/320.1

(57) ABSTRACT

A new IRES sequence derived from a plant gene that is enabling cap independent translation in eukaryotic cells. The IRES sequence is enabling stress induced translation.

### CROSS-REFERENCE TO RELATED APPLICATIONS

This application is a continuation of International Application Number PCT/EP01/01026 filed on Feb. 1, 2001, designating the United States of America, International Publication No. WO 01/59138 (Aug. 16, 2001), which itself claims priority to European Patent Application EP 00200442.2 filed on Feb. 10, 2000, the contents of both applications are incorporated herein by this reference.

### BACKGROUND

Technical Field: The present invention relates to a sequence capable of initiating cap independent translation. More particularly, the present invention relates to a sequence that is capable of initiation cap independent translation in plants.

The concept of eukaryotic translation initiation is primarily based on the interaction of a number of initiation factors (eIF's) and common cis-acting elements along eukaryotic mRNA's (5′-cap, poly A and AUG-context). These universal features emphasize the non-selective targeting of messengers by the cap-dependent translation (CDT) initiation process. This has important implications for cell protein synthesis in response to developmental and/or environmental changes. Under these conditions, when cells need to accumulate readily specific proteins, the CDT process has to be reduced and replaced by (or at least modified into) a translation initiation process that allows selective targeting of response-specific messengers. Sequence elements in the 5′ non-coding regions of eukaryotic messengers can initiate cap independent translation (CIT) by internal initiation of ribosomes. These internal ribosome entry sites (IRESs) were first found in uncapped picornaviral mRNA's (Pelletier and Sonenberg, 1988), but later also found in a limited amount of natural capped cellular messengers in yeast (Iizuka et al., 1994), mammals (Macejak and Sarnow, 1991; Vagner et al., 1995; Teerink et al., 1995; Gan and Rhoads et al., 1996; Bernstein et al., 1997; Nanbru et al., 1997; Stein et al., 1998) and Drosophila (Oh et al., 1992; Ye et al., 1997). It has been suggested that this alternative mechanism of translation could be used for the selective translation of mRNA's during growth, differentiation and in a wide variety of stress responses. IRESs containing messengers are often characterized by extremely long and highly structured leader sequences with multiple upstream AUGs (van der Velden and Thomas, 1999). Aside from a conserved oligopyrimidine tract at a fixed distance from the AUG start codon within the picornaviral 5′ UTRs (Pilipenko et al., 1992), there is little primary sequence conservation. Recently, it was shown that the plant translational apparatus is capable of exhibiting cap independent translation on viral IRESs (Skulachev et al., 1999), but contrary to the situation in mammalian genes, internal ribosome entry site (IRES) sequences have not been described in plant genes.

Translational control mediated by oligopyrimidine tracts in ribosomal protein (rp) genes has been established for many years. All vertebrate rp-mRNA's have a typical short 5′-UTR and start with a terminal oligopyrimidine (TOP) tract (Meyuhas et al., 1996). These leader sequences are necessary and sufficient for the upshift from ribonucleoproteins (RNP's) to polysomes to maintain the proper stoichiometry of the ribosomal components during rapid cell growth (Levy et al., 1991; Hammond et al., 1991; Patel and Jacobs-Lorena, 1992; Avni et al., 1994; Amaldi et al., 1995). Several reports suggest that the translational control of rp-genes is distinct from the cap dependent protein synthesis. Laurent et al. (1998) showed that the shut-off of host protein synthesis by herpes simplex virus type 1 (HSV-1) infection is controlled at the translation initiation step. However, HSV-1 infection did not affect the translation efficiency of mRNA's harbouring a 5′ TOP, like rp-genes (Simonin et al., 1997; Greco et al., 1997). Shama et al. (1995) demonstrated that the efficiency of translation of rp-mRNA is regulated independently of the level, the phosphorylation state or the activity of eIF-4E, the cap-binding component of the eIF-4F complex. Despite these data, internal initiation in a ribosomal protein mRNA has never been reported. Although S6 phosphorylation (Thomas and Thomas, 1986; Jefferies et al., 1994) and/or protein factors that bind 5′ TOP sequences (Kaspar et al., 1992; Pellizzoni et al., 1996) have been proposed as putative determinants in the regulation of translation of vertebrate rp-genes, the exact mechanism is still unknown. In plants, much less information is available on translational control mechanisms in rp-mRNA's. In this respect, two interesting observations were made by Shama and Meyuhas (1996): (i) the plant translational apparatus recognizes the 5′ TOP regulatory elements of mammalian rp genes and (ii) from an evolutionary point of view, translational control of rp genes precedes the appearance of the 5′ TOP, suggesting that translational cis-acting regulatory elements do not have to resemble a 5′ TOP. Of the eight plant nuclear rp genes that were fully examined by primer extension or nuclease protection assays, only one had a typical 5′ TOP sequence (Shama and Meyuhas, 1996).

A number of plant viral mRNAs are not capped and must have a cap-independent translation mechanism. Cap-independent translation might still be dependent on ribosome association with the RNA 5′ end and not involve a true IRES. Although sometimes reported in literature, the existence of IRESs on plant viral RNAs is not generally accepted and needs more substantiation (Fütterer and Hohn, 1996).

The formation of intermolecular complexes between eukaryotic animal mRNA's and the 18S rRNA has been demonstrated several times (Tranque et al, 1998; Hu et al., 1999). Basepairing between polypyrimidine tracts on viral mRNA's and purine-rich sequences in the 18S rRNA was often proposed as a model for ribosome recognition as the first step of CIT. A conserved UUUCC element (box A) in the polypyrimidine tract of picornaviral IRESs is fully complementary to the 3′-end of the 18S rRNA (Pillipenko et al., 1992). Similar models were suggested for novel cap-independent translation initiation events mediated by 3′-UTR translational enhancer sequences as in satellite tobacco necrosis virus (STNV) RNA (Danthinne et al., 1993; Meulewaeter et al., 1998) and PAV barley yellow dwarf virus (BYDV-PAV) RNA (Wang et al., 1997), but, although small sequence segments (up to 11 bp) complementary to the 18S rRNA are found routinely in eukaryotic messengers (Joshi and Nguyen, 1995) no evidence was found yet that these prokaryotic like interactions could lead to cap independent translation initiation in plant genes.

### DISCLOSURE OF THE INVENTION

Surprisingly, we found that the leader sequence of RPS18C, belonging to the Arabidopsis RPS18 gene family, contains an IRES and can initiate cap independent translation. Cap independent ribosome recognition was triggered by basepairing of a 5′ UTR oligopyrimidine tract to the 3′ end of the 18S rRNA. This sequence contains a motif that is similar to the “box A” of picornviral IRESs. The cap independent translation can be inhibited by the sequence shown in SEQ ID NO:1, which is complementary to the 3′ end of the 18S rRNA. Even more surprisingly, the cap independent translation is active and induced under stress conditions, preferably salt stress and/or general starvation.

One aspect of the present invention is to provide an isolated polynucleotide, enabling initiation of translation in an eukaryotic system, characterized by the fact that the initiation of translation and the subsequent translation can be inhibited by an oligonucleotide with SEQ ID NO:1. Another aspect of the invention is an isolated polynucleotide with IRES activity, enabling cap-independent initiation of translation in a eukaryotic system, wherein the isolated polynucleotide is derived from a plant gene, preferably not a heat shock protein gene. Still another aspect of the invention is an isolated polynucleotide, enabling cap-independent initiation of translation, wherein the polynucleotide may form a stable interaction with a sequence derived from the 3′ end of the plant 18S rRNA. The 3′ end as defined here comprises the last two hairpin loops, and may be considered as the last 170 nucleotides of the sequence (5762-5932 of genbank sequence accession number X52322). A preferred embodiment of the invention is an isolated polynucleotide, enabling cap-independent initiation of translation in an eukaryotic system, encoding a polynucleotide comprising the polynucleotide shown in SEQ ID NO:2, or the complement of the isolated polynucleotide. Preferentially, the eukaryotic system is a plant system. As the IRES activity, enabling cap-independent initiation of translation is based on the interaction of the mRNA sequence with the 18S rRNA, variations in SEQ ID NO:2 can be tolerated, as long as the interaction with the 18S rRNA is not disturbed. A typical example of such a variation is a U to C transition on position 6 and/or 11 of SEQ ID NO:2. Therefore, another preferred embodiment of the invention is an isolated polynucleotide, enabling cap-independent initiation of translation in an eukaryotic system, encoding a polynucleotide comprising the polynucleotide shown in SEQ ID NO:3, or the complement of the isolated polynucleotide. Preferentially, the eukaryotic system is a plant system.

Such cap-independent initiation of translation and subsequent translation may be used to create a dicistronic and/or oligocistronic expression systems. The construction and use of such expression systems in mammalian cells is well known to those skilled in the art and has been described in the international patent applications WO 94/05785, WO 96/01324 and WO 98/11241. It is an aspect of the invention to provide a novel IRES that may be used in the mammalian dicistronic and/or oligocistronic expression systems. It is another aspect of the invention to create dicistronic and/or oligocistronic expression systems for plant cells. Such system can be created by making a vector, suitable for transformation of plant cells, comprising

a suitable promoter sequence

a first coding sequence, preceded by a 5′ untranslated region with a normal cap structure

at least one IRES according to the invention, followed by another coding sequence

Another aspect of the invention is an isolated plant polynucleotide, enabling initiation of translation in a eukaryotic system, preferentially a plant cell, wherein the initiation of translation is induced by stress conditions. Preferably, the stress is salt stress and/or general starvation. Even more preferably, the stress induced initiation of translation and subsequent translation can be inhibited by an oligonucleotide with SEQ.ID.N^{o}1. Such stress-induced initiation of translation may be used as an alternative for a stress induced promoter. Indeed, when the plant polynucleotide is produced by a constitutive promoter or by a promoter that allows transcription under the stress conditions, the translation of a coding sequence placed after the plant polynucleotide will be induced under stress conditions. Constitutive promoters are known to those skilled in the art.

The stress inducible IRES can be placed in front of the coding sequence that one wants to express during stress conditions, such as a coding sequence providing stress resistance. These coding sequences are known to those skilled in the art and include, but are not limited to, superoxide dismutase, heat shock proteins or proteins conferring salt resistances such as, for plants, Arabidopsis thaliana Sos3p.

Although the stress inducible IRES may be used as an alternative for stress induced transcription, it may also be used in combination with a stress inducible promoter. As it is known that cap dependent translation is affected in a negative way by stress, the combination stress inducible promoter/stress inducible IRES will result in a higher protein production—and in case of the use of a coding sequence providing stress protection, a concomitant higher stress protection—than when the stress inducible promoter alone is used.

Another aspect of the invention is an isolated polynucleotide, preferably DNA, encoding a polynucleotide, preferably RNA, enabling initiation of translation in an eukaryotic system, characterized by the fact that the initiation of translation and the subsequent translation can be inhibited by an oligonucleotide with SEQ ID NO:1 and/or characterized by the fact that the initiation of translation is induced by stress conditions. A preferred embodiment is an isolated DNA fragment encoding a RNA fragment comprising SEQ ID NO:2, or the complement of the DNA fragment. Another preferred embodiment is an isolated DNA fragment encoding a RNA fragment comprising SEQ ID NO:3, or the complement of the DNA fragment.

Still another aspect of the invention is a transformation vector, comprising the DNA fragment or polynucleotide.

A further aspect of the invention is an eukaryotic cell, transformed with the transformation vector. Particular embodiments are a transgenic plant or a transgenic animal, transformed with the transformation vector.

Another aspect of the invention is a method for facilitating cap independent translation of mRNA in an eukaryotic cell by incorporating a DNA fragment, encoding a RNA fragment capable of initiating translation in an eukaryotic system, before a coding sequence, wherein the initiation of translation is characterized by the fact that the initiation of translation and the subsequent translation can be inhibited by an oligonucleotide with SEQ ID NO:1. In a preferred embodiment of the invention, the RNA fragment comprises SEQ ID NO:2 or SEQ ID NO:3.

Still another aspect of the invention is a method for facilitating stress induced translation in a eukaryotic cell by incorporating a DNA fragment, encoding a RNA fragment capable of initiating stress-induced translation before a coding sequence. A preferred embodiment is said method, whereby said stress-induced initiation of translation and the subsequent translation can be inhibited by an oligonucleotide with SEQ.ID.N^{o}1. Another preferred embodiment is said method, whereby said RNA fragment comprises SEQ.ID.N^{o}2.

Definitions

The following definitions are set forth to illustrate and define the meaning and scope of the various terms used to describe the invention herein:

Polynucleotide as used herein refers to a polymeric form of nucleotides of any length, either ribonucleotides or deoxyribonucleotides. This term refers only to the primary structure of the molecule. Thus, this term includes double and single-stranded DNA, and double or single stranded RNA. It also includes known types of modifications, for example methylation, cap structure, and substitution of one or more of the naturally occurring nucleotides with an analog.

IRES or IRES sequence is a polynucleotide that enables initiation of translation and subsequent translation when placed in front of an appropriate sequence, containing a start coding and an open reading frame. The translation is cap independent and may start anywhere in the mRNA. IRES sequences are especially useful for the construction of multicistronic messenger RNAs.

Enabling initiation of translation as used herein means that the polynucleotide which is enabling the initiation of translation may function as a control sequence for translation, either directly, as part of the mRNA, or indirectly, as part of the DNA that is transcribed into RNA. The translation enabled by an IRES sequence is cap independent. The term initiation of translation refers to the first steps of translation, including the binding of the ribosomal subunits to the messenger RNA. As used here, the initiation of translation implies that, when the control sequence is placed upstream of a suitable coding sequence, the initiation of translation is followed by translation of the coding sequence. Therefore, the initiation of translation may be checked in an in vitro translation system, such as a wheat germ system, by using an oligonucleotide comprising the control sequence upstream of a suitable coding sequence, and checking either protein synthesis or polysome formation.

Eukaryotic system means any eukaryotic cell, eukaryotic organism or eukaryotic based cell free transcription and/or translation system and comprises therefore both in vitro and in vivo systems. In particular, eukaryotic system means, but is not limited to, a plant cell, a plant, an animal cell, an animal, a yeast or fungal cell, wheat germ extract and rabbit reticulocyte lysate.

Transformation vector means any vector, known to those skilled in the art, capable of transforming an eukaryotic cell. It includes, but is not limited to replicative vectors and integrative vectors, Agrobacterium based transformation vectors and viral vector systems such as retroviral vectors, adenoviral vectors or lentiviral vectors.

Inhibition of translation means that there is a decrease of 40%, preferentially 60%, more preferentially 100% of in vitro protein synthesis by adding 100 pmoles inhibitor, compared to the non-inhibited situation, as measured in a Wheat Germ in vitro translation system. As an example, a Wheat Germ in vitro translation system is described below. Alternatively, other Wheat Germ in vitro translation systems, known to those skilled in the art, may be used. In vitro translation reactions are carried out using 3 pmoles in vitro synthesized RNA, in the presence of Rnasin Ribonuclease Inhibitor (Promega), with final concentrations of 73 mM potassium acetate and 2.1 mM magnesium acetate in Wheat Germ (Promega). In vitro translation products are labelled with Amersham International Redivue L-[³⁵S] methionine and analyzed after 45 min reaction time. Proteins are separated on 12% polyacrylamide gels, fixed in 10% acetic acid, treated with Amersham's Amplify, dried and quantified using a Molecular Dynamics PhosphorImager and ImageQuant 4.1 software.

Gene as used herein means the regions of the DNA that can be transcribed into RNA in an eukaryotic cell when the DNA is linked to a promoter functional in the eukaryotic cell. The RNA is preferentially, but not necessarily translated into protein. In case the RNA is translated into protein, the term gene includes the 5′ end and 3′ end untranslated regions. In that respect, DNA encoding a gene as used herein means the DNA fragment from the start of transcription until the end of transcription.

Plant polynucleotide means a fragment that is originally part of a genomic plant gene or encoded by a genomic plant gene, even if this polynucleotide is produced in another host cell than a plant cell.

Stress conditions mean all kind of stress, known to those skilled in the art and include, but are not limited to heat shock, osmotic stress, salt stress, oxygen stress and starvation.

Stress induced translation as used herein means that the translation is still active under stress conditions. It includes both a real induction of the translation, i.e., a situation where there is no translation of the coding sequence in absence of stress conditions, but translation in the presence of stress conditions, as well a relative induction of the translation, i.e. a comparable efficiency of translation in stress conditions and in absence of stress conditions, whereas the other messengers are less efficiently translated in stress conditions.

### BRIEF DESCRIPTION OF THE SEVERAL VIEWS OF THE DRAWINGS

FIG. 1. Expression analysis of the RPS18 genes. (A) Schematic representation of the position of the RT-PCR primers (black arrows) on the mRNAs. Open and closed triangles represent intron positions in all three genes and the T-DNA insertion in RPS18A, respectively. (B) Quantitative RT-PCR kinetics: graphical time course representing the kinetics of the three PCR reactions within one sample (roots in this case). Under the given conditions, the different samples were quantified in the linear phase of the reaction (18 cycles). (C) Absolute quantities at 18 cycles of the different RPS18 genes in wild-type tissues (lane 1, roots; lane 2, 5-day-old plants; lane 3, 12-day-old plants; lane 4, 19-day-old plants; lane 5, flowering plants; lane 6, young leaves; lane 7, mature leaves) compared to the pointed first leaves (pfl) mutant (lane 8, mature leaves). (D) Presentation of the experiment described in (C) on a polyacrylamide gel.

FIG. 2. Complementary sequences in the RPS18C leader to the 3′ end of the 18S rRNA and secondary structures. (A) Primary sequence of the RPS18C leader (including the translation start codon) showing the complementary sequence to the 18S rRNA (bold and uppercase) (SEQ ID NO:24). (B) Primary and secondary structure of the 3′ end of the 18S rRNA from positions 1645 to 1803, (SEQ ID NO:25) showing the potential interaction site with the RPS18C leader (bold and uppercase). Underlined bases indicate the repetitive GGAAGG motif. (C) The 15-bp complementary region between mRNA_{—}15 and rRNA_{—}15 (12 Watson-Crick base pairs and 3 G-U wobble pairs) (SEQ ID NO:26). The arrow shows the junction between the stem-loop sequence and the freely accessible bases in the 18S rRNA. (D) The predicted secondary structures of the RPS18C leader, including 24 bp of coding sequence (SEQ ID NO:22) (SEQ ID NO:23). The complementary sequences to 18S rRNA are marked by black circles. Boxed sequences show the position of the duplex formed by a GAAGA motif with either an downstream UCUUC or a upstream element (structure I and structure II, respectively), as shown in-between both structures (AUG start codon is indicated by three asterisks).

FIG. 3. RNA oligonucleotide competition experiments in a wheat germ translation system. (A) Oligo#1 (SEQ ID NO: 16) and random oligo (GAUCGAUCGAUC) (SEQ ID NO:19). (B) Oligo#2 (SEQ ID NO:17). (C) Oligo#3 (SEQ ID NO:18). (D) Oligo#4 (SEQ ID NO:1). nts, nucleotides. Encircled bases on the secondary structure of the 18S rRNA show the interaction site of the complementary sequences in the oligonucleotides. The protein gels and graphs show the competition effect of increasing amounts of the RNA oligonucleotide, measured by the translation efficiency of uncapped RPS18Cleader/CAT as a percentage relative to 100% (no oligonucleotide). Dotted lines in all graphs represent the random oligonucleotide, black lines in all graphs represent the effect of the respective oligonucleotides.

FIG. 4. (A) The competition effect of oligo#1 on the translation of the four viral proteins (109,94,35 and 20 kD) of Brome Mosaic Virus (BMV) RNA (lanes 1 and 2) compared to the RPS18Cleader/CAT RNA (lanes 4 and 5) (+, addition of 136 pmoles of oligo#1;−, no oligo control) (lane 3: 0, no RNA control). (B) Competition assay on RPS18Cleader/CAT with increasing amounts of oligo#4, showing the translation products (upper part) and the intact transcripts after Northern analysis on the same samples (lower part). (C) Some polysome profiles of the reactions done in FIG. 4B, after fractionation of a linear 10% to 45% sucrose density gradient. After centrifugation fractions were collected from bottom of the tube and measured at 260 nm. 1: negative control (no RNA, with 272 pmoles oligo#4); 2: RPS18Cleader/CAT transcript (without oligo#4); 3:RPS18Cleader/CAT (with 272 pmoles oligo#4).

FIG. 5. (A) Translation in Wheat Germ of RPS18Cleader/CAT: O: no RNA; C−: without Cap and C+: with Cap. (B) Dicistronic reporter constructs harboring the luciferase (LUC)-coding sequence as the first ORF and chloramphenicol acetyltransferase (CAT) as the second ORF. LUC/−/CAT is the negative control. The LUC/RPS18Cleader/CAT construct bears the leader of RPS18C fused at the AUG start codon of CAT. (C) In vitro translation of equimolar amounts of discistronic constructs in wheat germ (left) and translation in rabbit reticulocyte lysates (right) using ³⁵S-methionine and polyacrylamide gel separation. Arrows show a band, interfering with CAT, derived from the LUC ORF. (D) Northern analysis after translation of the dicistronic transcripts showing intact non degraded RNA in Wheat Germ (to the left) and in rabbit reticulocyte lysates (to the right). Lane 1: no transcript; lane 2: LUC/−/CAT and lane 3:LUC/RPS18Cleader/CAT.

FIG. 6. (A) Oligonucleotide-directed mutagenesis on the LUC/RPS18Cleader/CAT (SEQ ID NO:27). Mutated bases are marked by asterisks (SEQ ID NO:28) (SEQ ID NO:29). Single stranded and stemloop sequences of the rRNA_{—}15 are indicated below the mRNA_{—}15 mutagenized sequences (B) CAT assays on TLC. bCm, butyrylated chloramphenicol; Cm, chloramphenicol; ori, origin. Lane 1, Ω/CAT; lane 2, no transcript; lane 3, LUC/−/CAT; lane 4, LUC/RPS18Cleader/CAT; lane 5, LUC/#110/CAT; lane 6, LUC/#111/CAT; lane 7, LUC/#112/CAT. (C) Graphical representation of the results from the CAT assays relative to Ω/CAT set to 100% (same lanes as in B).

### DETAILED DESCRIPTION OF THE INVENTION

### EXAMPLES

### Example 1

### Expression Analysis of the RPS18 Genes

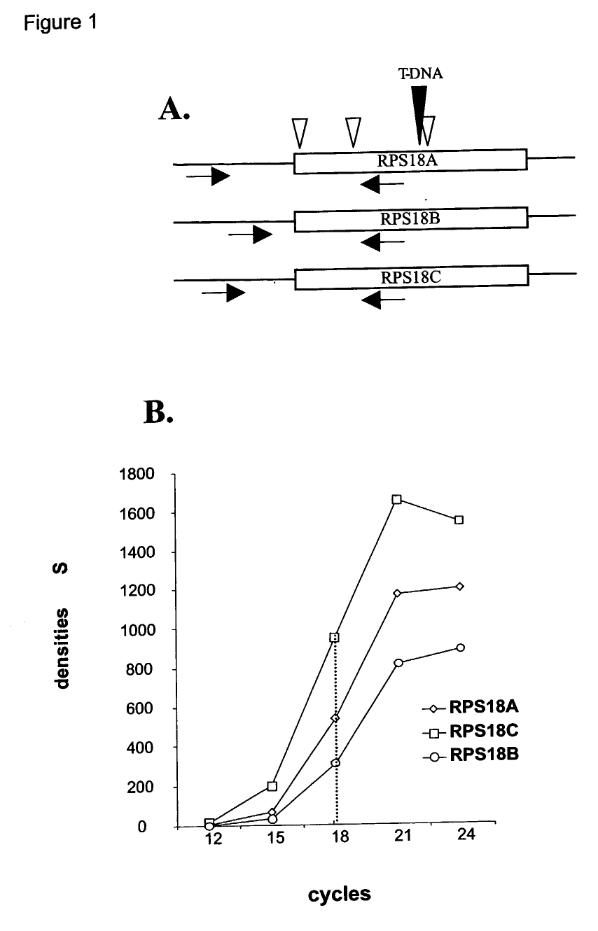

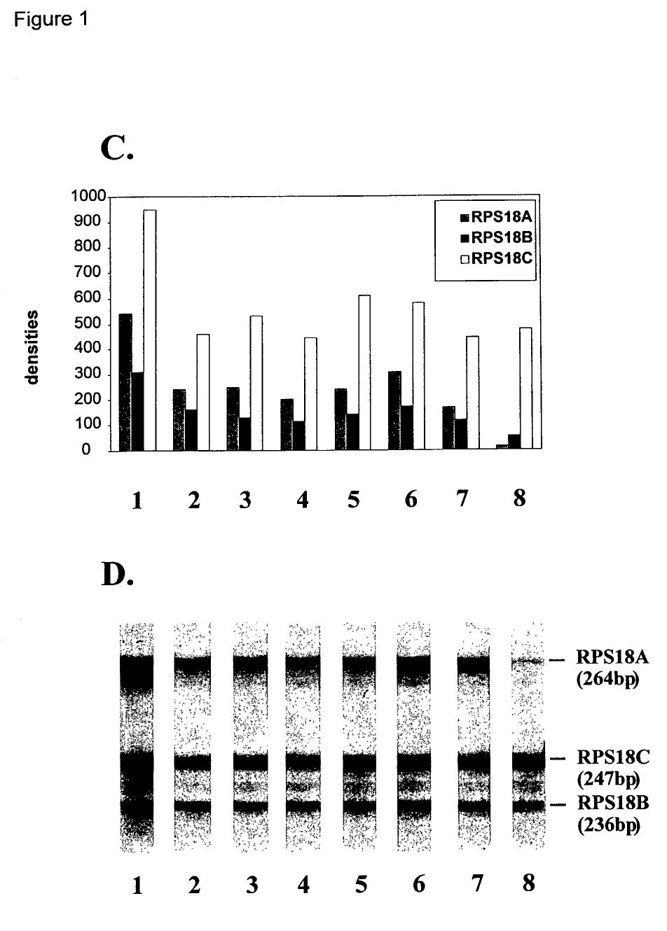

The Arabidopsis ribosomal protein S18 is encoded by three expressed genes. A T-DNA insertion in the RPS18A gene caused the pfl (pointed first leaves) phenotype, and is the only mutation described in an eukaryotic S18 protein (Van Lijsebettens et al., 1994). Besides an alteration of the shape of the first leaves, it causes growth retardation and an overall 20% reduction in biomass. This moderate phenotype was proposed to be the result of a reduction in the total amount of synthesized S18 protein in mutant cells. This would imply that trancriptional control mechanisms in the two other genes, to upregulate the pool of S18 mRNA, are absent. To study the transcriptional contribution of the three gene copies in different tissues from wild type plants compared to the pfl mutant, a multiplex RT-PCR system was set up using three gene-specific primers in the 5′-UTR region in combination with a common kinated primer in the coding sequence (FIG. 1, part A). Isolation of mRNA was done according to protocol using the QuickPrep® mRNA Purification Kit (Pharmacia Biotech). The glycogen precipitation step allows mRNA purification from tissue as little as a single embryo. In the latter case, an embryo was pushed out of the seed under the binocular and tranferred, after 5 consecutive washes in RNase-free water, to the extraction buffer and homogenized by sonication in a Misonix XL2020 (Branson, Genève, Switzerland). cDNA synthesis was performed using Superscript™ Preamplification System for First Strand cDNA Synthesis (Gibco/BRL, Gaithersburh, Md.). The PCR was done using three gene-specific primers in the 5′ UTR of the different RPS18 genes in combination with a common kinated primer in a conserved sequence in the coding region of the third exon (RPS18A: 5′ TGGTGGCGCCTCCAGAGTCTGG 3′ (SEQ ID NO:4); RPS18B: 5′ TTCTCAGGCATCTCTTATCTTC 3′ (SEQ ID NO:5); RPS18C: 5′ ACGGCTTCTTCTTCTCACAA 3′ (SEQ ID NO:6); common primer: 5′ GTCATGAGGTTATCAATCTCAG 3′ (SEQ ID NO:7)). The common primer had the lowest thermodynamic values (Tm=44.1° C.; G=−35.4 kcal/mol) and determines the PCR kinetics in all three reactions to the same extent. The PCR cycle parameters were: 1 min 94° C., 30 sec 52,5° C. and 30 sec 72° C. in conditions described in Van Lijsebettens et al., 1994. PCR products were analyzed on polyacrylamide sequencing gels, dried and exposed overnight and quantified using a Molecular Dynamics PhosphorImager and ImageQuant 4.1 software (Molecular Dynamics, Sunnyvale, Calif.).

The PCR products were analyzed at a fixed time point during the linear phase of the reaction (FIG. 1, part B); the results are shown in FIG. 1 part D. The resulting densities of the bands are representative of the initial concentration of the different transcripts in the samples. The relative quantities of the three transcripts in the different tissues were remarkably stable (FIGS. 1 part C and 1 part D, lanes 1 to 7). The contribution to the pool of messengers coding for the S18 protein in wild type Arabidopsis plants was on average 27% for the RPS18A copy, 16% for the B copy and 57% for the C copy. Even in actively-dividing tissue, such as a heart stage embryo, no significant difference in this ratio could be found. Furthermore, the analysis of the pfl mutant revealed the molecular mechanism leading to this aberrant phenotype. The level of expression of the RPS18A transcript in the mutant, relative to the two other copies, was reduced to 3% (FIGS. 1 part C and 1 part D, lane 8). This loss of functional transcripts causes a shortage of S18 protein that alters the proper stoichiometry of ribosomal components. As a result, mutant cells fail to produce a sufficient amount of functional ribosomes. This affects total protein synthesis, resulting in a slower growth rate and reduced fresh weight of the plant. Implicit in this concept is that both other genes are not upregulated at the transcriptional level. FIGS. 1 part C and 1 part D (lanes 7 and 8) show indeed, that the level of expression of the RPS18B and C gene in the pfl mutant are comparable to the wild type, indicating total absence of any feedback mechanism from the S18 protein to these genes to accumulate its mRNA. These results indicate that a general regulatory control mechanism for the coordinate synthesis of r-proteins in plants is not acting at the transcriptional level, as in yeast, but possibly at the translational level, as in vertebrates, invertebrates and Dictyostelium.

### Example 2

### Plasmid Construction

□/CAT is similar to pFM169 (Meulewaeter et al., 1998) and is basically a T7/SP6 in vitro transcription vector, where the TMV leader is fused to the CAT coding region followed by a poly(A) sequence. RPS18Cleader/CAT was made by a translational fusion of the RPS18C leader to the CAT coding region in pFM169. In a first step the leader sequence was amplified by PCR from a RPS18C genomic clone using primers: 5′ CCTCTTTTGGGATCCTCACTCTC 3′ (SEQ ID NO:8) and 5′ CTAATTACCATGGTGATTAGCAGAG 3′ (SEQ ID NO:9), hereby creating a BamHI and NcoI (underlined in the primers) restriction site at the 5′ end of the leader and at the AUG-startcodon, respectively. In a similar step, the NH2-terminal-part of CAT was amplified from pFM169 using primers: 5′ ACTATTCTAGCCATGGAGAA 3′ (SEQ ID NO:10) and 5′ CCATACGGAATTCCGGATGA 3′ (SEQ ID NO:11), introducing a NcoI site at translation startcodon of CAT and covering the existing EcoRI site at position 286 in pFM169, respectively. Both fragments were purified, cut and ligated. The resulting BamHI/EcoRI-fragment was cloned in pFM169 cut with the same enzymes. Selected clones were sequenced and checked for correct sequence integrity. The monocistronic LUC construct used in this work derived from the pT3/T7-Luciferase Expression Vector ordered at Clontech Laboratories, Inc. (Palo Alto, Calif.).

The LUC/RPS18Cleader/CAT construct was made by inserting a 1.9 kb BamHI-fragment from pT3/T7 LUC, covering the entire Luciferase gene, in front of RPS18Cleader/CAT and cut with BamHI. The negative control LUC/−/CAT was made by inserting the blunt-ended BamHI-Luciferase fragment in the blunt-ended SacI site of pFM136 (Meulewaeter et al., 1992), that is basically a pGEM-3Z vector containing the CAT coding region. The poly(A) sequence from pFM169 was inserted as an XbaI-HindIII-fragment behind the CAT coding region of LUC/−/CAT.

The sequence context around the AUG startcodon was heavily modified during the RPS18Cleader/CAT fusion. We restored the original AUG context sequence, as in the RPS18C-mRNA, by an oligo directed mutagenesis using the pAlter-1 vector system from Promega (Madison, Wis.), following the protocol as described by the manufacturer. The oligo used was: 5′ CGATCTGGAATTAAAATGTCTAAAAAAATCACTGG 3′ (SEQ ID NO:12) showing the original bases (underlined) around the translation start (italic). As a consequence, the second amino acid in the CAT protein sequence became a serine instead of glutamic acid, but this change had no effect on CAT activity.

### Example 3

### In vitro Transcription and Translation

In vitro RNA synthesis of all constructs, except pT3/T7 LUC, was carried out on 1 μg HindIII-linearized DNA-templates using T7 RNA polymerase as described in the protocol of the Ampliscribe High Yield Transcription Kit supplied by Epicentre Technologies (Madison, Wis.). pT3/T7 LUC was linearized with SmaI using T3 RNA polymerase to produce run-off transcripts. Capped transcripts were made according to the protocol using the cap analog, m⁷G(5′)ppp(5′)G, from Pharmacia. After DNaseI treatment the RNA was purified by precipitation, according to the manufacturer's recommendation, with one volume 5M ammonium acetate followed by centrifugation, a 70% ethanol wash and dissolved in RNase-free water. The RNA-quality was checked by agarose gel electrophoresis and the RNA was quantified by a Beckmann DU-64 spectrophotometer (Beckmann instruments, London, UK) after mixing thoroughly.

In vitro protein synthesis was performed in Wheat Germ as well as in Rabbit Reticulocyte Lysate (R.R.L.) Systems from Promega. In vitro translation reactions were done using 3 pmoles in vitro synthesized RNA, in the presence of RNasin Ribonuclease Inhibitor (Promega), with final concentrations of 73 mM potassium acetate and 2.1 mM magnesium acetate in Wheat Germ and respectively 79 mM and 1.4 mM in Rabbit Reticulocyte Lysates. In vitro translation products were labeled with Amersham International Redivue L-[³⁵S]methionine (Amersham, Aylesbury, UK) and analyzed after 45 min reaction time. Protein products were separated on 12% polyacrylamide gels, fixed in 10% acetic acid, treated with Amersham's Amplify, dried and quantified using a Molecular Dynamics PhosphorImager and ImageQuant 4.1 software. Alternatively (as in the mutagenesis experiments), CAT translational products were analyzed using the CAT Enzyme Assay System from Promega with a thin layer chromatography (TLC) assay. The CAT assays were performed on total R.R.L.-reactions for 20 hours. TLC-plates were exposed during 48 hours and the predominant band of the butyrylated chloramphenicol (bCm) isoforms was quantified as described above. All results were reproduced at least twice.

### Example 4

### Sequences in the RPS18C Leader are Complementary to the 3′ End of the 18S rRNA

In plants, most ribosomal proteins are encoded by multiple gene copies. 5′ TOP-like sequences, as in vertebrates, are not common but most of the plant rp genes have internal oligopyrimidine tracts (IOTs) in their 5′ UTR. The RPS18A gene has not an IOT, the RPS18B gene copy has a 5′ IOP, the RPS18C gene has both.

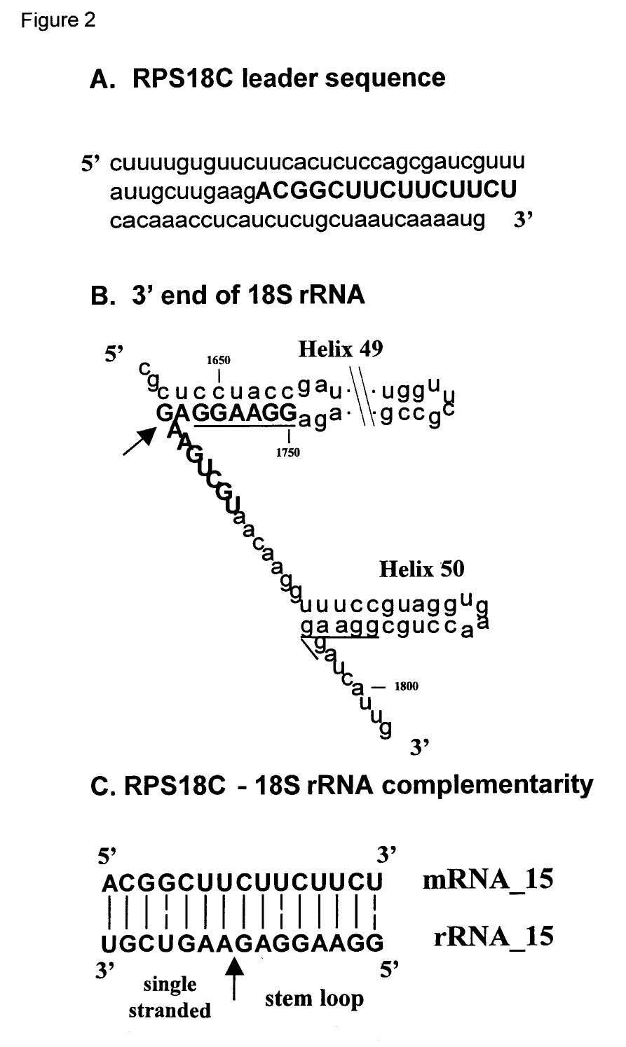

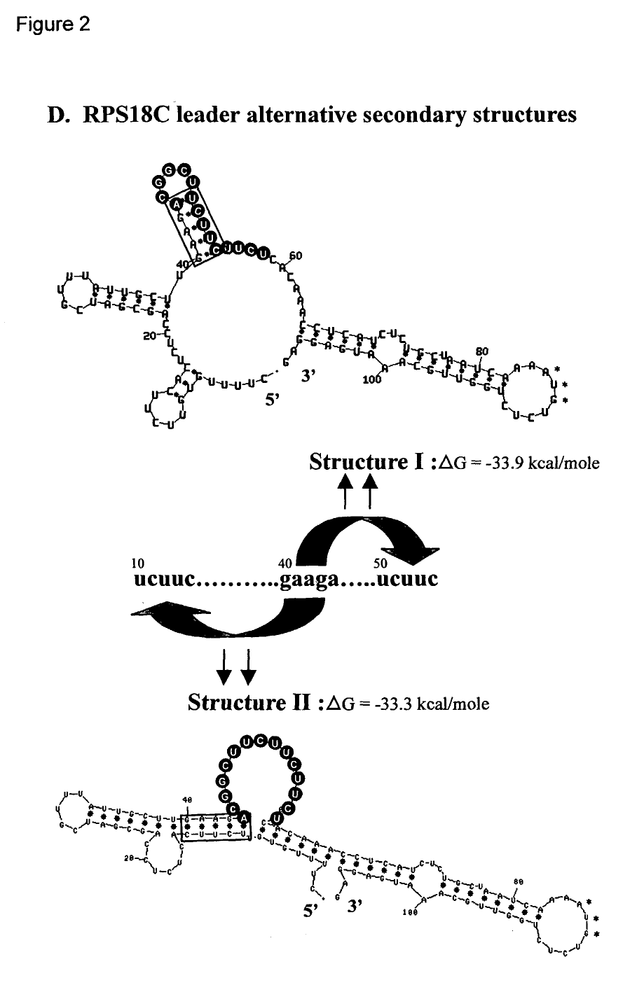

The RPS18C leader (FIG. 2, part A), contains an IOT (11 bp) localized in a stretch of 15 nucleotides at position 44 to 58 (defined as mRNA_{—}15 in FIG. 2, part C). mRNA_{—}15 is fully complementary to a region near the 3′ end of the Arabidopsis thaliana 18S rRNA sequence at position 1750 to 1764 (defined as rRNA_{—}15 in FIG. 2, part C). The last eight bases match to a GA-rich sequence within the stem of helix 49, whereas the first seven bases match to single-stranded sequences between helices 49 and 50 according to the 18S rRNA three-dimensional model described by Van de Peer et al. (2000) (FIGS. 2, part B and 2, part C). Although this region in the 18S rRNA is highly conserved in eukaryotes, it was never proposed as a putative interaction site with mRNAs. Interestingly, it contains a GGAAGG motif that is repeated further downstream in the 18S rRNA from positions 1791 to 1796 (underlined in FIG. 2, part B). At this site, the motif has been proposed several times as the functional analog of the anti-Shine-Dalgarno region of prokaryotes (Dolph et al., 1990; Pilipenko et al., 1992; Danthinne et al., 1993; Wang et al., 1997).

Secondary structure analysis of the RPS18C leader predicts two different configurations with comparable free-energy values (FIG. 2, part D). The RNA secondary structure of the RPS18C leader was calculated using the mfold program version 2.3 at 22° C. (Zuker et al., 1999; http://mfold2.wustl.edu/˜mfold/rna/form1.cgi). The predictions resulted in two alternative configurations (FIG. 2, part D), which clearly remained identical in the temperature range from 10 to 35° C. Below 22° C., the predictions on structure II showed two extra base-pairing events GG/CU at positions 46-47/54-55 within the complementary region. Secondary structure predictions of LUC/RPS18Cleader/CAT and the mutant dicistronic constructs in the intercistronic region were done on the constructs including at least 50 bp of sequences flanking the RPS18C leader. Free-energy parameters and melting temperatures (based on the nearest neighbor method) of the oligonucleotides used here were calculated using the OLIGO 4.0 software (Rychlik et al., 1990).

Remarkably, in both structures, the initiating AUG localizes in a very strong stem loop structure that might affect the ribosome scanning process. Both structures are basically the same but differ in the folding of the mRNA_{—}15 sequence. In structure I, the mRNA_{—}15 folds partially into a stem by the pairing of a UCUUC element to an upstream GAAGA element. In structure II, an upstream UCLUC element can form a duplex with this GAAGA motif, leaving the mRNA_{—}15 sequence single stranded. This prediction shows that the transition from one configuration to the other is easy and would influence the accessibility of the mRNA_{—}15.

### Example 5

### RNA Oligonucleotide Competition Experiments Suggest an Intermolecular Interaction between the RPS18C Leader and the 3′ end of the 18S rRNA

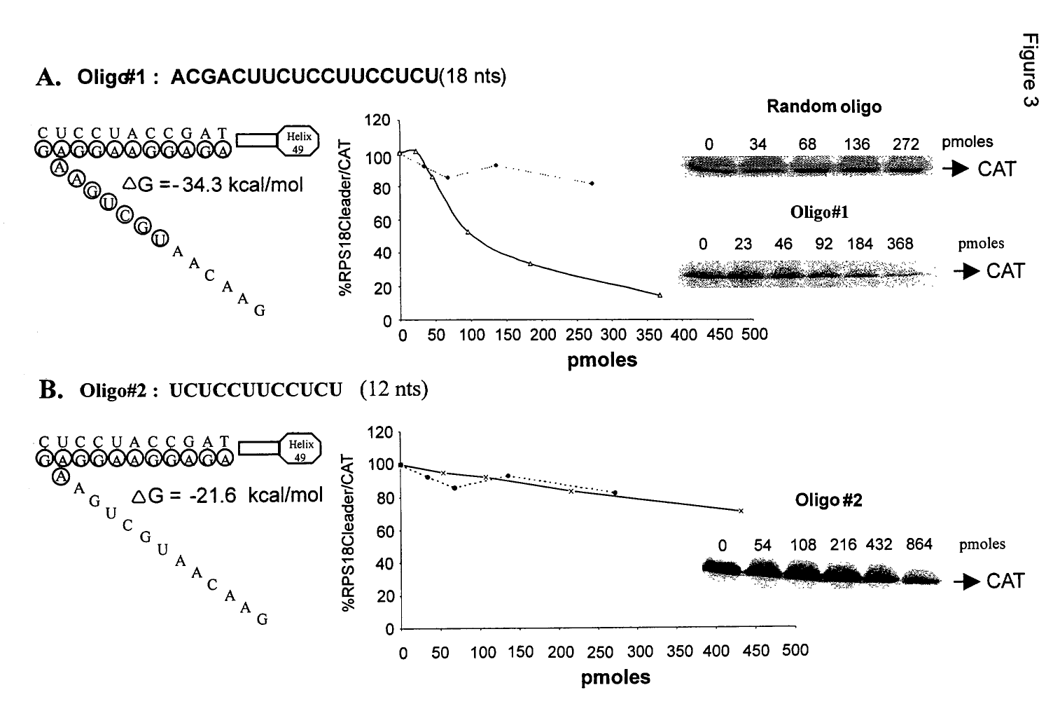

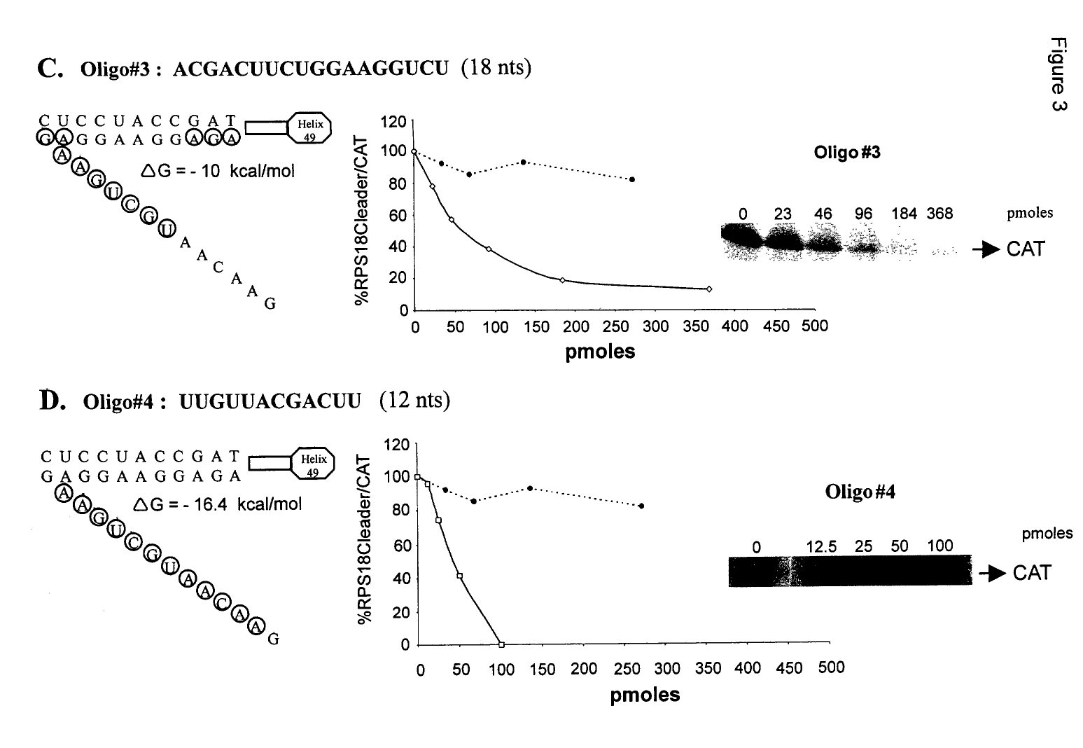

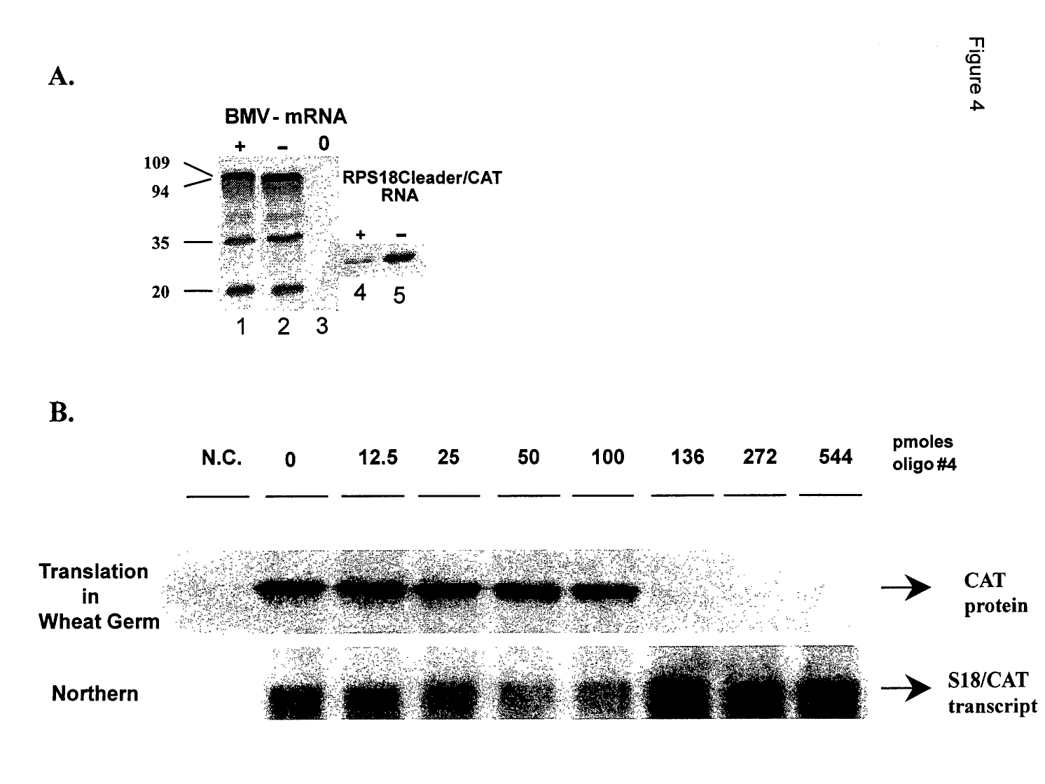

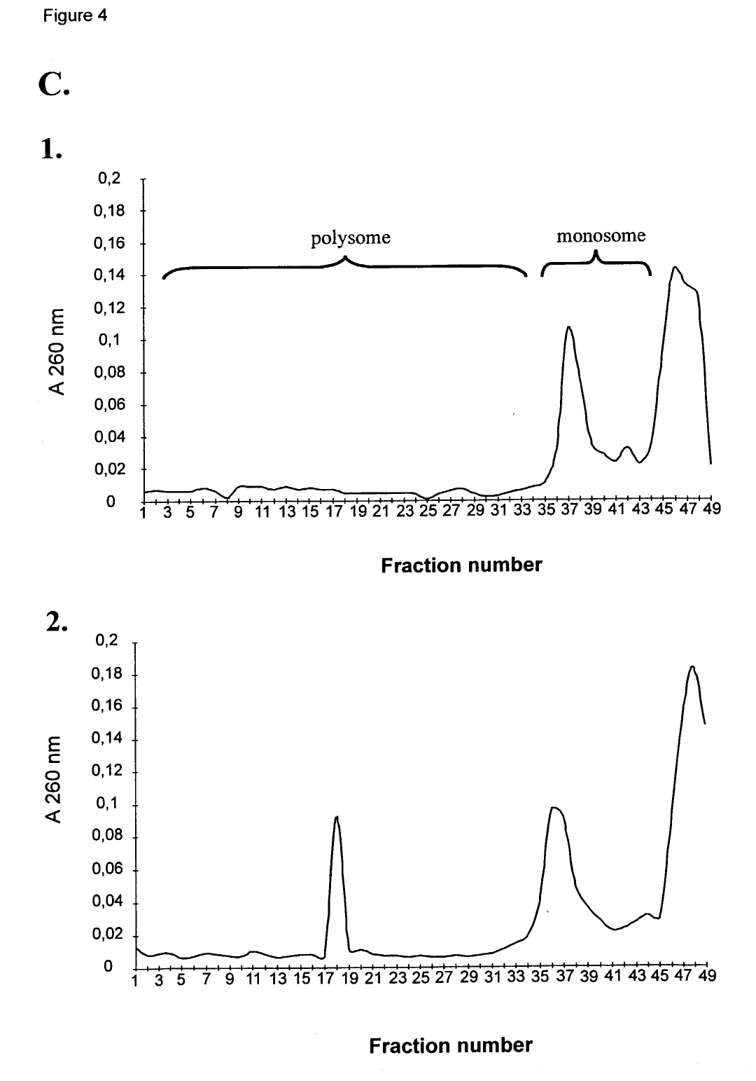

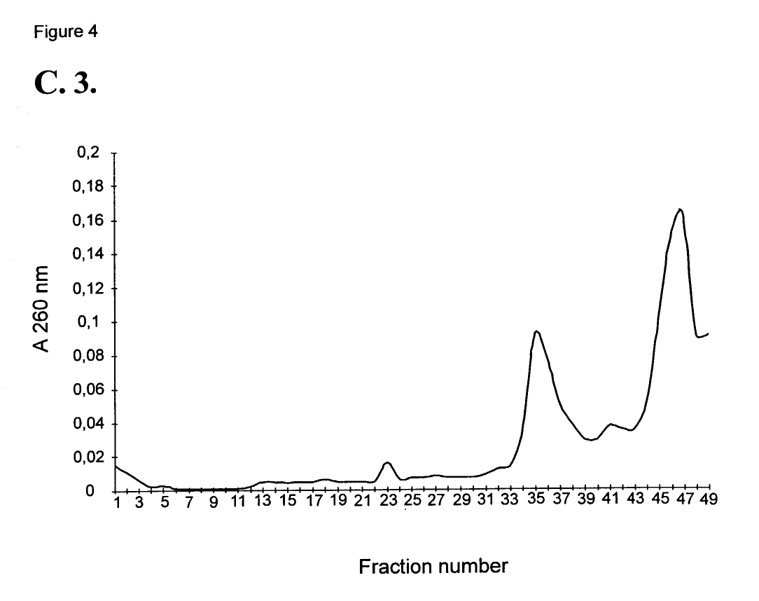

A 15-bp sequence motif has a very low probability to occur in the Arabidopsis genome (even considering the three GU base pairs). A search with rRNA_{—}15 in the Arabidopsis genome database only showed similarity to 18S rRNA-related sequences. The exceptionally long stretch of complementary sequences in the RPS18C leader suggests that intermolecular interactions with the 18S rRNA might occur. To address the existence and the role of this interaction, we designed translation competition experiments between different RNA oligonucleotides and the uncapped RPS18C leader using a cell-free translation system. We fused the RPS18C leader to the chloramphenicol acetyltransferase (CAT) reporter gene (RPS18Cleader/CAT) and measured translation efficiencies in wheat germ upon addition of increasing amounts of the different oligonucleotides. The RNA oligonucleotides were purchased from Genset (Paris, France) (sequences are shown in FIG. 3), diluted in RNase-free water, and checked for their concentration. All tests were repeated 2 to 4 times and were done with the same batch of wheat germ extract and starting from master mixtures to avoid variation of translational components within the samples. FIG. 3A shows that oligo#1, fully complementary to positions 1747 to 1764 of the 3′ end of the 18S rRNA, reduced the translation efficiency of the uncapped RPS18Cleader/CAT by 50% at approximately 100 pmoles. In contrast, equimolar amounts of a random oligonucleotide (GAUCGAUCGAUC) (SEQ ID NO: 19) had no effect. This suggests that oligo#1 and the mRNA_{—}15 sequences were competing for the same site on the 18S rRNA sequence. We further compared the competition effects of oligonucleotides complementary within the stem of helix 49 of the 18S rRNA and oligonucleotides complementary to the freely accessible strand outside the stem (FIGS. 3, part B, 3, part C, and 3, part D). Oligo#2, a 12-nt oligonucleotide fully complementary inside the stem of helix 49, showed no competitive effect compared to oligo#3, which had complementary sequences outside the stem. The free energy value for duplex formation of oligo#2 was much higher than that of oligo#3 (−21.6 kcal/mol and −10 kcal/mol, respectively), indicating that the smaller size of oligo#2 was not responsible for this effect. On the other hand, oligo#3 (lacking the CCUUCC internal stem loop complementary sequences) had a competitive effect comparable to that of oligo#1 (FIGS. 3, part C and 3, part A), although its free-energy value was much lower (−10 kcal/mol and −34.3 kcal/mol for oligo#3 and oligo#1, respectively). These results suggest that initially the interaction with mRNA_{—}15 occurs at the freely accessible part of the rRNA_{—}15. Translation of RPS18Cleader/CAT rapidly dropped to zero when using oligo#4, which is complementary to the complete single-stranded sequence between helices 49 and 50 on the 18S rRNA (FIG. 3, part D). The higher amount of complementary sequences and, consequently, the faster recruitment of oligo#4 to the 18S rRNA is presumably the reason for this effect. The specificity and the integrity of these oligo competition assays were confirmed by a set of experiments summarized in FIG. 4. First, the oligonucleotides that inhibit the translation of RPS18Cleader/CAT had no effect (in similar conditions) on the translation of brome mosaic virus (BMV) transcripts that are naturally capped, illustrated for oligo#1 in FIG. 4, part A. Secondly, the intactness of the RNA, after translation, was verified by Northern analysis as shown for oligo #4 in FIG. 4, part B. Northern analysis was carried out as described before (Van Lijsebettens et al., 1994). Samples were extracted twice with phenol/chloroform and precipitated with one volume of 5 M ammonium acetate before loading onto formamide gels. A CAT-DNA probe was used to identify the transcripts.

For practical reasons we used a higher initial concentration of RPS18Cleader/CAT transcripts compared to FIG. 3, part D, affecting the range but not the slope of the competition assay. Interestingly, less full-length transcripts could be detected in the samples where translation was not blocked, assuming that, after phenolisation of the translation reactions, the transcripts associated to the polysomal fraction were extracted from the sample. Thirdly, FIG. 4C shows the polysome profiles of three samples from FIG. 4, part B: no transcript (FIG. 4, part C1), RPS18Cleader/CAT transcript without oligo (FIG. 4, part C2) and RPS18Cleader/CAT with 272 pmoles oligo#4 added (FIG. 4, part C3). The polysome profiles were attained by loading 15 μl of the reaction mixture after translation onto a linear 10% to 45% sucrose density gradient in 25 mM Tris.HCl (pH: 7.6), 100 mM KCl and 5 mM MgCl₂. After centrifugation in a SW41 rotor (Beckmann) at 38000 rpm for 1 hour at 4° C., fractions were collected from the bottom of the tubes and were measured at 260 nm. A high yield of polysomes could only be detected upon translation of RPS18Cleader/CAT in absence of an inhibiting amount of the competing oligo. These data clearly indicate that oligo#4 blocks translation by preventing polysome assembly.

The RNA competition experiments showed that the 5′ end of the mRNA_{—}15 sequence (ACGACUU) (SEQ ID NO:20), referred to as the “activator” sequence has an important function in initiating mRNA-rRNA contact by a standby mechanism.

### Example 6

### RPS18C Leader Allows Cap-independent Translation in vitro

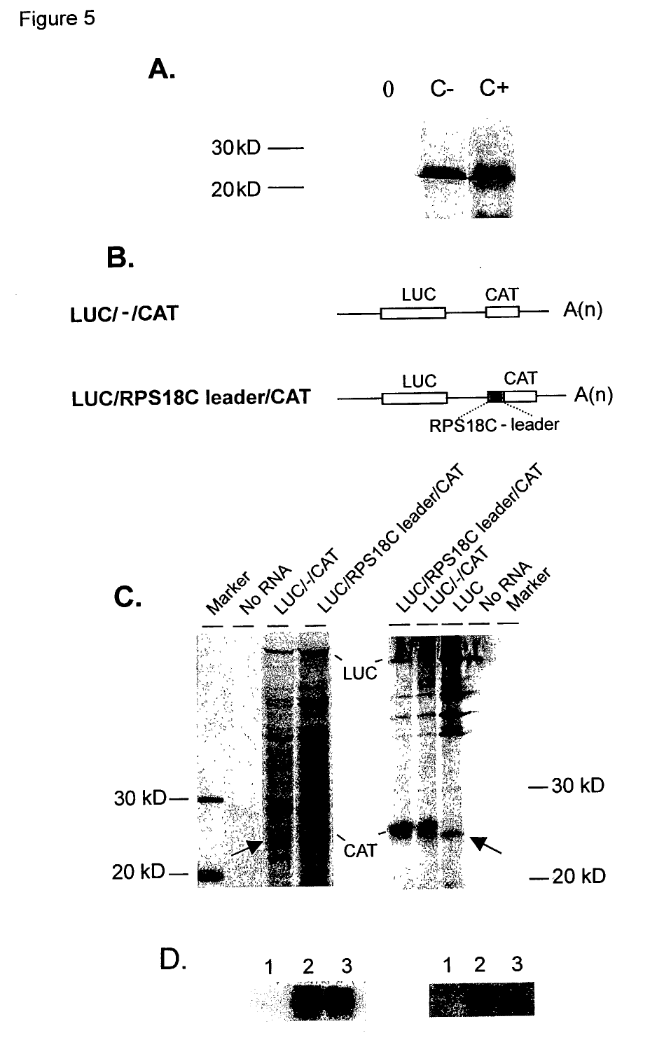

Direct interaction of RPS18C with the 3′ end of the 18S rRNA could be an alternative mechanism to circumvent the cap dependent translation initiation process. This hypothesis was supported by the fact that the translation of uncapped RPS18Cleader/CAT transcripts was very efficient in Wheat Germ, and the addition of a cap-analog to these transcripts did not improve the translation efficiency significantly (FIG. 5, part A). Internal entry of ribosomes on sequences in eukaryotic cells can be established by translating dicistronic mRNA's (Jackson, 1996). Insertion of the internal ribosome binding site between two open reading frames (ORF's) stimulates the translation of the second ORF. As described above, the dicistronic vector LUC/RPS18Cleader/CAT was constructed using the luciferase (LUC) coding sequence as the first ORF, and the CAT coding sequence preceded by RPS18C leader as the second one (FIG. 5, part B). As a negative control, LUC/−/CAT, in which the CAT coding region is situated immediately adjacent to the 3′ UTR of LUC, was used (FIG. 5, part B). Translation of the second ORF was enhanced in LUC/RPS18Cleader/CAT compared to the negative control LUC/−/CAT, in wheat germ (FIG. 5, part C, left panel) as well as in rabbit reticulocyte lysates (R.R.L.) (FIG. 5, part C, right panel). The translation of the second cistron in the LUC/RPS18Cleader/CAT transcripts was more efficient than the first one in both translation systems but more explicit in Rabbit Reticulocyte Lysates. The consideration that the larger LUC protein sequence can incorporate five 35S labeled methionine residues per molecule more than CAT, emphasizes this observation. Remarkably, the efficient translation of the second ORF from LUC/RPS18Cleader/CAT transcripts (in R.R.L) reduces the translation of the first ORF (compare lanes 1 and 2 in FIG. 5, part C, right panel) showing the autonomy of the translation of the second ORF. The fact that more CAT is produced than LUC can not be explained by a readthrough mechanism nor by fragmentation of the transcripts, since the intactness of the RNA's were validated after translation, by Northern analysis (FIG. 5, part D). In all tests with the LUC/−/CAT transcripts there was still a low amount of CAT present, probably caused by leaky scanning or ribosome readthrough. In both translation systems a protein band appeared that interfered with CAT. This band represents a smaller translation product coming from the luciferase coding sequence since it was detected in the translation of monocistronic LUC transcripts (indicated by an arrow, FIG. 5, part C).

These results indicate that ribosomes can enter internally on the leader sequence of the RPS18C gene and translate a heterologous transcript. This implies that RPS18Cleader contains an autonomous IRES element, the first one described in a plant cellular messenger RNA.

### Example 7

### Cis-acting Sequences in RPS18C Leader are Involved in Cap-independent Translation

Interaction with the 18S rRNA and internal entry of ribosomes are two unique features of the RPS18C leader. To verify whether both processes are linked to the same site on the leader sequence, the RPS18C leader in the LUC/RPS18Cleader/CAT dicistronic construct was mutagenized and the effect on the internal translation of CAT was studied by measuring CAT activity. The mutagenesis was performed using the Altered Sites II in vitro Mutagenesis Systems from Promega. The pAlter-1 vector system was used following the protocol as described by the manufacturer. The dicistronic construct LUC/RPS18Cleader/CAT served as mutagenesis template and the respective mutagenic oligonucleotides to make LUC/#110/CAT, LUC/#111/CAT and LUC/#112/CAT were as follow: #110, 5′ GTTTATTGCTTGAAGTGCTGAACTTCTTCTCAC 3′ (SEQ ID NO:13); #111, 5′ GCTTGAAGACGGCTTGAGGAAGGCACAAACCTCATCT 3′ (SEQ ID NO:14) and #112, 5′ ACGGCTTCTTGAACTCACAAACCTC 3′ (SEQ ID NO:15). The original bases were substituted by their counterparts as they occur in the 18S rRNA (underlined). All mutant clones were sequenced before use.

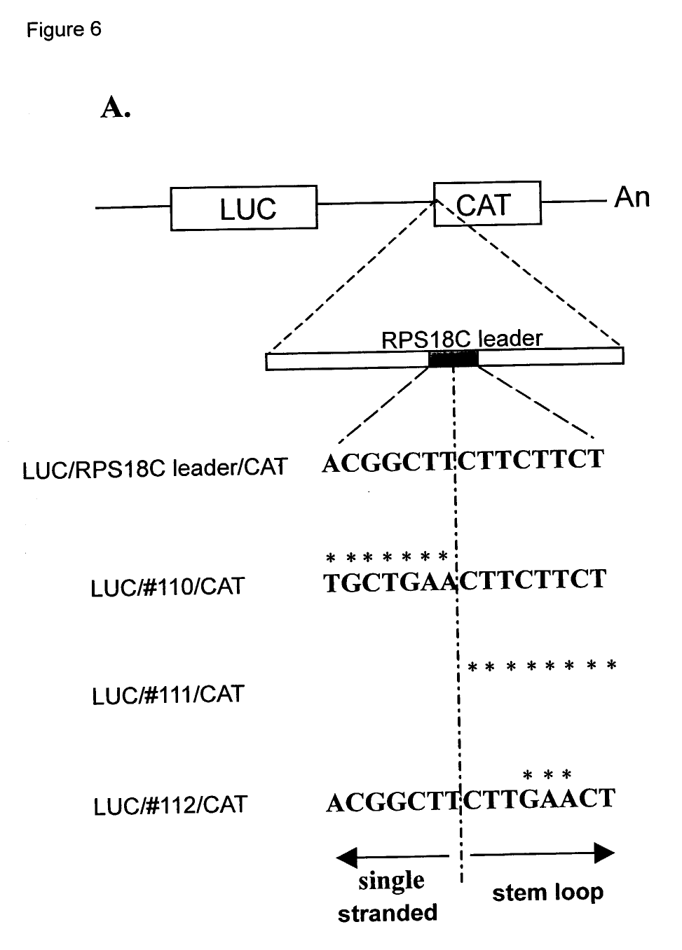

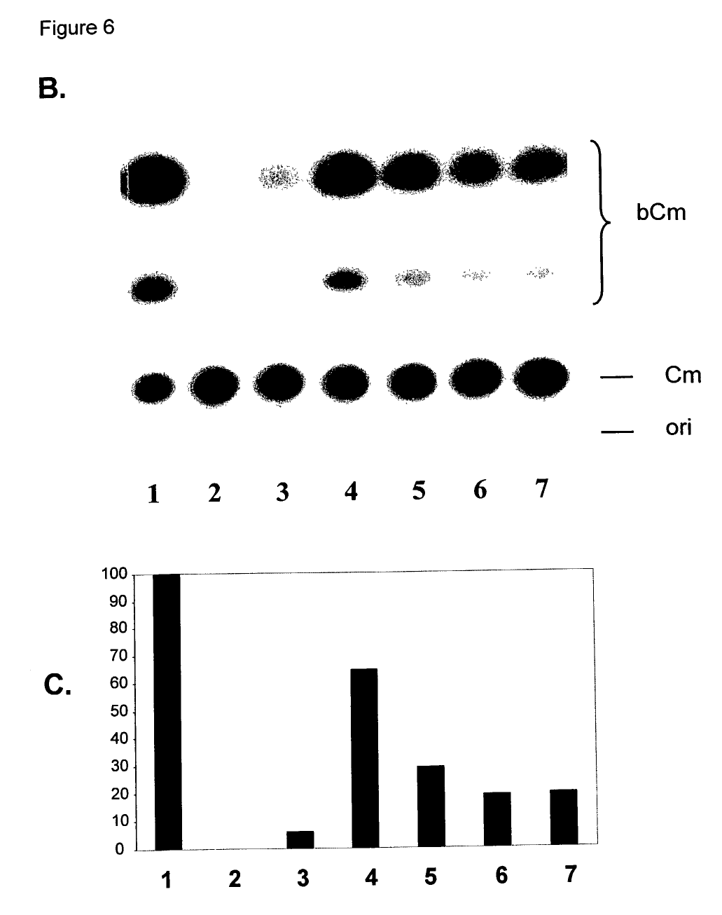

Since proper folding of the RNA was proposed to play an important role for the activity of picornavirus IRES elements (Pillipenko et al., 1992), for each mutant construct the effect on secondary structure is shown (FIG. 6, part A). The RNA secondary structure of the RPS18C leader and the effects of the different mutations on secondary structure were calculated with RNAdraw (Matzura and Wennborg, 1996). Mutations were made by substituting the original bases by their complementary bases on the 18S rRNA (FIG. 6, part A). ). In the LUC/#110/CAT construct, we replaced the bases complementary to the freely accessible sequence (the activator sequence). LUC/#111/CAT was made by substituting the eight bases complementary to the stem sequences of helix 49. In the third mutant construct, LUC/#112/CAT, three bases (CUU) were changed in the core region (CUUCU) that showed homology to the picornavirus box A (UUUC(C)) (Pilipenko et al., 1992) and the translational enhancer domain (TED) motif (CUUCC) in STNV (Danthinne et al., 1993). The predicted secondary structure in the intercistronic regions of LUC/RPS18Cleader/CAT, LUC/#110/CAT and LUC/#112/CAT is quite similar to that in the RPS18C leader (FIG. 2, part D) specially as far as the region between the pyrimidine tract and the AUG start codon is concerned. This is not the case in the LUC/#111/CAT construct where an additional stem loop is predicted between both motifs. The efficiency of translation of CAT in LUC/RPS18Cleader/CAT was compared to a monocistronic transcript in which CAT was under control of the Ω leader of the tobacco mosaic virus (Ω/CAT), described as a translational enhancer (Gallie and Walbot, 1992). Comparison of the CAT activities of LUC/RPS18Cleader/CAT and Ω/CAT showed that the efficiency of internal initiation was 35% lower than that of a monocistronic transcript under control of a translational enhancer. These results indicate that this plant IRES is a very efficient cap-independent translation system.

Translation of the LUC/#110/CAT transcript reduced CAT activity by 55% compared to LUC/RPS18Cleader/CAT (FIGS. 5, part B and 5, part C, lanes 4 and 5). Translation of the LUC/#112/CAT mutant transcript was reduced by 70% (FIGS. 6, part B and 6, part C, lane 7), indicating that the activator sequence is not the essential element for internal initiation. A comparable decrease in CAT activity could be observed with LUC/#111/CAT and LUC/#112/CAT (FIGS. 6B and 6C, lanes 6 and 7), indicating that the picornavirus box A-like motif is an important element for internal initiation at this IRES. Indeed, a 3-bp change in the CUUCU core region was sufficient to reduce the function of the IRES significantly. Therefore, the CUUCUUCU tract (complementary to the stem sequences of helix 49) will be referred to as the “effector” sequence. No additionally reducing effects were seen in the translation of LUC/#111/CAT, compared to LUC/#112/CAT.

### Example 8

### RPS18C Leader-IRES is Stress Regulated in Vivo

A 311 bp EcoRI fragment comprising the unique BamHI site, the RPS18C leader and the NH2 terminal part of CAT was cut from pLUC/RPS18Cleader/CAT, gel purified and made blunt end by Klenow. On the other hand pGUS1 was cut with NcoI at the translation startcodon and filled in by Klenow. Both blunt ended fragments were ligated resulting in the plasmid pRPS18Cleader/CAT/GUS1 bearing an in frame fusion of the NH2 terminal region of CAT with the coding region of gus. PRPS18Cleader/CAT/GUS1 was cut BamHI-XbaI and made blunt end by Klenow, generating a fragment containing the complete RPS18Cleader/CAT/GUS fusion including the 3′OCS UTR. This fragment was cloned in the blunt ended SpeI site of pAPPGfp200201 (kindly provided by Elena Babiychuk). APPGfp expresses a translational fusion of poly ADP ribose polymerase of Arabidopsis thaliana and Gfp. This fusion is targeted to the nucleus. pAPPGfp200201 is a T-DNA vector with the backbone of pGSV6 and the hygromycine resistance cassette of pHYG661 between the T-DNA borders. The resulting bicistronic construct in pAPPGfp/RPS18Cleader/GUS is under control of the 35S promoter and has APPGfp as the first ORF and an in frame fusion of the amino terminal part of CAT with gus as the second ORF preceded by the RPS18Cleader-IRES.

Tobacco BY2 cells were transformed with pAPPGfp/RPS18Cleader/GUS according to the method of Shaul et al (1996). Transformants were selected by hygromycine resistance and individual clones were analysed by fluorescent microscopy for GFP expression. GFP positive lines were grown in liquid BY2 medium at 28° C. in different conditions and analysed by histochemical staining with X-Gluc as substrate according to Jefferson et al. (1987).

Eight days old liquid BY2 cultures were subsampled (0.5 ml in 50 ml fresh medium) and grown in different stress conditions (heat: 43° C., salt: 200 mM NaCl, starvation: medium without sucrose and general starvation: 14 days old overgrown cultures). An indigo blue precipitate could be visualized by dark field microscopy in all cells 24 to 72 hours after staining with X-Gluc in conditions of salt stress and general starvation.

It is inherent to the mode of interaction with the 18S rRNA that the RPS18C-IRES would function very inefficiently in cells under normal growth conditions. The amount of free cytoplasmic 40S subunits that are available for this interaction is very low compared to those that are assembled into the polysomes. Consequently, RPS18C-IRES activity might increase considerably when the normal cap-dependent translation process is reduced or shut-off and the proportion of free 40S subunits increases, as in stress conditions or during mitosis. We screened the plant sequence database for genes with box A-like motifs in the same context as in the RPS18C leader. The PatScan software (http://www-unix.mcs.anl.gov/compbio/PatScan/HTML/patscan.html) was used to look for the presence of a motif in the 5′ UTR of plant mRNAs at an arbitrary distance (10 to 100 nucleotides) from a translation start codon in the consensus context (RHRAUG). To reduce the amount of data, the motif used was essentially the complete CU tract as it occurs in the RPS18C leader (CUUCUUCUUCU) (SEQ ID NO:2), covering the complete effector sequence extended at the 5′ end with CUU (from the activator sequence). At two positions (CUUCUYCUUCY) (SEQ ID NO:3), variations were allowed because cytosines at these positions would cause an even stronger binding to the 18S rRNA. Only expressed and fully annotated genes were considered in this search

Interestingly, 50% of the hits represented genes involved in stress response. These data also indicate that the RPS18C-IRES is stress regulated, similar to the tightly regulated IRESs in cellular mRNAs (Stein et al., 1998; Johannes et al., 1999)

### REFERENCES

Amaldi, F., Camacho-Vanegas, O., Cardinall, B., Cecconi, F., Crosio, C., Loreni, F., Mariottini, P., Pellizzoni, L. and Pierandrei-Amaldi, P. (1995) Structure and expression of ribosomal protein genes in Xenopus laevis. Biochem. Cell. Biol., 73, 969-977.

Avni, D., Shama, S., Loreni, F. and Meyuhas, O. (1994) Vertebrate mRNAs with a 5′-terminal pyrimidine tract are candidates for translational repression in quiescent cells: characterization of the translational cis-regulatory element. Mol. Cell. Biol., 14, 3822-3833.

Bernstein, J., Sella, O., Le, S. Y. and Elroy-Stein, O. (1997) PDGF2/c-sis mRNA leader contains a differentiation-linked internal ribosomal entry site (D-IRES). J. Biol. Chem., 272, 9356-9362.

Danthinne, X., Seurinck, J., Meulewaeter, F., Van Montagu, M. and Cornelissen, M. (1993) The 3′ untranslated region of satellite tobacco necrosis virus RNA stimulates translation in vitro. Mol. Cell. Biol., 13, 3340-3349.

Dolph, P. J., Huang, J. T. and Schneider, R. J. (1990) Translation by the adenovirus tripartite leader: elements which determine independence from cap-binding protein complex. J. Virol., 64, 2669-2677.

Fütterer, J. and Hohn, T. (1996) Translation in plants—rules and exceptions. Plant Molecular Biology, 32, 159-189.

Gallie, D. R. and Walbot, V. (1992) Identification of the motifs within the tobacco mosaic virus 5′-leader responsible for enhancing translation. Nucleic Acids Res., 20, 4631-4638.

Gan, W. and Rhoads, R. E. (1996) Internal initiation of translation directed by the 5′-untranslated region of the mRNA for eIF4G, a factor involved in the picornavirus-induced switch from cap-dependent to internal initiation. J. Biol. Chem., 271, 623-626.

Greco, A., Laurent, A. M. and Madjar, J. J. (1997) Repression of beta-actin synthesis and persistence of ribosomal protein synthesis after infection of HeLa cells by herpes simplex virus type 1 infection are under translational control. Mol. Gen. Genet., 256, 320-327.

Hammond, M. L., Merrick, W. and Bowman, L. H. (1991) Sequences mediating the translation of mouse S16 ribosomal protein mRNA during myoblast differentiation and in vitro and possible control points for the in vitro translation. Genes Dev., 5, 1723-1736.

Hu, M. C., Tranque, P., Edelman, G. M. and Mauro, V. P. (1999) rRNA-complementarity in the 5′ untranslated region of mRNA specifying the Gtx homeodomain protein: evidence that base-pairing to 18S rRNA affects translational efficiency. Proc. Natl. Acad. Sci. USA, 96, 1339-1344.

Iizuka, N., Najita, L., Franzusoff, A. and Sarnow, P. (1994) Cap-dependent and cap-independent translation by internal initiation of mRNAs in cell extracts prepared from Saccharomyces cerevisiae. Mol. Cell. Biol., 14, 7322-7330.

Jackson, R. J. (1996) A comparative view of initiation site selection mechanisms. In Hershey, J. W. B., Mathews, M. B. and Sonenberg, N. (eds). Translational Control (Monograph Series, Vol. 30). Cold Spring Harbor Laboratory Press, Cold Spring Harbor, N.Y., pp. 71-113.

Jefferies, H. B., Reinhard, C., Kozma, S. C. and Thomas, G. (1994) Rapamycin selectively represses translation of the “polypyrimidine tract” mRNA family. Proc. Natl. Acad. Sci. USA. 91, 4441-4445.

Jefferson, R. A., Kavanagh, T. A. and Bevan, M. W. (1987) GUS fusions: β-glucuronidase as a sensitive and versatile gene fusion marker in higher plants. EMBO J., 6, 3901-3907.

Johannes, G., Carter, M. S., Eisen, M. B., Brown, P. O. and Sarnow, P. (1999). Identification of eukaryotic mRNAs that are translated at reduced cap binding complex eIF4F concentrations using a cDNA microarray. Proc. Natl. Acad. Sci. USA, 96, 13118-13123.

Joshi, C. P. and Nguyen, H. T. (1995° 5′ untranslated leader sequences of eukaryotic mRNAs encoding heat shock induced proteins. Nucl. Acids Res., 23, 541-549.

Kaspar, R. L., Kakegawa, T., Cranston, H., Morris, D. R. and White, M. W. (1992) A regulatory cis element and a specific binding factor involved in the mitogenic control of murine ribosomal protein L32 translation. J. Biol. Chem., 267, 508-514.

Laurent, A. M., Madjar, J. J. and Greco, A. (1998) Translational control of viral and host protein synthesis during the course of herpes simplex virus type 1 infection: evidence that initiation of translation is the limiting step. .J Gen. Virol., 79, 2765-2775.

Levy, S.; Avni, D., Hariharan, N., Perry, R. P. and Meyuhas, O. (1991) Oligopyrimidine tract at the 5′ end of mammalian ribosomal protein mRNAs is required for their translational control. Proc. Natl. Acad. Sci. USA, 15, 3319-3323.

Macejak, D. G. and Sarnow, P. (1991) Internal initiation of translation mediated by the 5′ leader of a cellular mRNA. Nature, 353, 90-94.

Matzura, O. and Wennborg, A. (1996) RNAdraw: an integrated program for RNA secondary structure calculation and analysis under 32-bit Microsoft Windows. Computer Applications in the Biosciences (CABIOS), 12(3), 247-249.

Meulewaeter, F., Cornelissen, M. and Van Emmelo, J. (1992) Subgenomic RNAs mediate expression of cistrons located internally on the genomic RNA of tobacco necrosis virus strain A. J. Virol., 66, 6419-6428.

Meulewaeter, F., Van Montagu, M. and Cornelissen, M. (1998) Features of the autonomous function of the translational enhancer domain of satellite tobacco necrosis virus. RNA, 4, 1347-1356.

Meyuhas, O., Avni, D. and Shama, S. (1996) Translational control of ribosomal protein mRNAs in eukaryotes. In Hershey, J. W. B., Mathews, M. B. and Sonenberg, N. (eds), Translational Control. Cold Spring Harbor Laboratory Press, Cold Spring Harbor, N.Y., pp. 363-388.

Nanbru, C., Lafon, I., Audigier, S., Gensac, M. C., Vagner, S., Huez, G. and Prats, A. C. (1997) Alternative translation of the proto-oncogene c-myc by an internal ribosome entry site. J. Biol. Chem., 272, 32061-32066.

Oh, S. K., Scott, M. P. and Sarnow, P. (1992) Homeotic gene Antennapedia mRNA contains 5′-noncoding sequences that confer translational initiation by internal ribosome binding. Genes Dev., 6, 1643-1653.

Patel, R. C. and Jacobs-Lorena, M. (1992) Cis-acting sequences in the 5′-untranslated region of the ribosomal protein A1 mRNA mediate its translational regulation during early embryogenesis of Drosophila. J. Biol. Chem., 267, 1159-1164.

Pelletier, J. and Sonenberg, N. (1988) Internal initiation of translation of eukaryotic mRNA directed by a sequence derived from poliovirus RNA. Nature, 334, 320-325.

Pellizzoni, L., Cardinali, B., Lin-Marq, N., Mercanti, D. and Pierandrei-Amaldi, P. (1996) A Xenopus laevis homologue of the La autoantigen binds the pyrimidine tract of the 5′ UTR of ribosomal protein mRNAs in vitro: implication of a protein factor in complex formation. J. Mol. Biol., 259, 904-915.

Pilipenko, E. V., Gmyl, A. P., Maslova, S. V., Svitkin, Y. V., Sinyakov, A. N. and Agol, V. I. (1992) Prokaryotic-like cis elements in the cap-independent internal initiation of translation on picornavirus RNA. Cell, 68, 119-131.

Rychlik, W., Spencer, W. J. and Rhoads, R. E. (1990) Optimization of the annealing temperature for DNA amplification in vitro. Nucleic Acids Res., 18, 6409-6412

Shama, S., Avni, D., Frederickson, R. M., Sonenberg, N. and Meyuhas, O. (1995) Overexpression of initiation factor eIF-4E does not relieve the translational repression of ribosomal protein mRNAs in quiescent cells. Gene Expression, 4, 241-252

Shama, S. and Meyuhas, O. (1996) The translational cis-regulatory element of mammalian ribosomal protein mRNAs is recognized by the plant translational apparatus. J. Biochem., 236, 383-388.

Shaul, O., Mironov, V., Burssens, S., Van Montagu, M. and Inze, D. (1996). Two Arabidopsis cyclin promoters mediate distinctive transcriptional oscillation in synchronized tobacco BY-2 cells. Proc. Natl. Acad. Sci. USA, 93, 4868-4872.

Simonin, D., Diaz, J. J., Masse, T. and Madjar, J. J. (1997) Persistence of ribosomal protein synthesis after infection of HeLa cells by herpes simplex virus type 1. J. Gen. Virol., 78, 435-443.

Skulachev, M. V., Ivanov, P. A., Karpova, O. V., Korpela, T., Rodionova, N. P., Dorokhov, Y. L. and Atabekov, J. G. (1999) Internal initiation of translation directed by the 5′-untranslated region of the tobamovirus subgenomic RNA I(2). Virology, 263, 139-154.

Stein, I., Itin, A., Einat, P., Skaliter, R., Grossman, Z. and Keshet, E. (1998) Translation of vascular endothelial growth factor mRNA by internal ribosome entry: implications for translation under hypoxia. Mol. Cell Biol., 18, 3112-3119.

Teerink, H., Voorma, H. O. and Thomas, A. A. (1995) The human insulin-like growth factor II leader 1 contains an internal ribosomal entry site. Biochim. Biophys. Acta, 1264, 403-408.

Thomas, G. and Thomas, G. (1986) Translational control of mRNA expression during the early mitogenic response in Swiss mouse3T3 cells: Identification of specific proteins. J. Cell Biol., 103, 2137-2144.

Tranque, P., Hu, M. C., Edelman, G. M. and Mauro, V. P. (1998) rRNA complementarity within mRNAs: a possible basis for mRNA-ribosome interactions and translational control. Proc. Natl. Acad. Sci. USA, 95, 12238-12243.

Vagner, S., Gensac, M. C., Maret, A., Bayard, F., Amalric, F., Prats, H. and Prats, A. C. (1995) Alternative translation of human fibroblast growth factor 2 mRNA occurs by internal entry of ribosomes. Mol. Cell Biol., 15, 35-44.

Van de Peer, Y., De Rijk, P., Wuyts, J., Winkelmans, T. and De Wachter, R. (2000) The European Small Subunit Ribosomal RNA database. Nucleic Acids Res., 28, 175-176.

van der Velden, A. W. and Thomas, A. A. M. (1999) The role of the 5′ untranslated region of an mRNA in translation regulation during development. Int. J. Biochem. Cell Biol., 31, 87-106.

Van Lijsebettens, M., Vanderhaeghen, R., De Block, M., Bauw, G., Villarroel, R. and Van Montagu, M. (1994) An S18 ribosomal protein gene copy at the Arabidopsis PFL locus affects plant development by its specific expression in meristems. EMBO J., 13, 3378-3388.

Wang, S., Browning, K. S. and Miller, W. A. (1997) A viral sequence in the 3′-untranslated region mimics a 5′ cap in facilitating translation of uncapped mRNA. EMBO J., 16, 4107-4116.

Ye, X., Fong, P., Iizuka, N., Choate, D. and Cavener, D. R. (1997) Ultrabithorax and Antennapedia 5′ untranslated regions promote developmentally regulated internal translation initiation. Mol. Cell Biol., 17, 1714-1721.

Zuker, M., Mathews, D. H. and Turner, D. H. (1999) Algorithms and thermodynamics for RNA secondary structure prediction: A practical guide. In Barciszewski, J. and Clark, B. F. C. (eds), RNA Biochemistry and Biotechnology, (NATO ASI Series 3, Vol. 70). Kluwer Academic Publishers, Dordrecht, The Netherlands, pp 11-43.

What is claimed is:

1. An isolated polynucleotide enabling initiation of translation in an eukaryotic system characterized by the fact that said initiation of translation and the subsequent translation can be inhibited by an oligonucleotide with SEQ. ID. NO: 1.

2. An isolated plant polynucleotide enabling cap independent initiation of translation in an eukaryotic system.

3. An isolated polynucleotide enabling initiation of translation in an eukaryotic system according to claim 1 or 2 whereby said isolated polynucleotide encodes a polynucleotide comprising SEQ. ID. NO: 2, or the complement of said isolated polynucleotide.

4. An isolated polynucleotide enabling initiation of translation in an eukaryotic system according to claim 1 or 2 whereby said isolated polynucleotide encodes a polynucleotide comprising SEQ. ID. NO: 3, or the complement of said isolated polynucleotide.

5. An isolated polynucleotide enabling initiating of translation in an eukaryotic system according to claim 1 to 4, whereby said eukaryotic system is a plant system.

6. An isolated plant polynucleotide, enabling stress induced initiation of translation.

7. An isolated plant polynucleotide according to claim 6, whereby said stress is salt stress and/or general starvation.

8. An isolated polynucleotide according to any of the claims 1, 3, 4 or 5, enabling stress induced initiation of translation.

9. An isolated polynucleotide according to claim 8, whereby said stress is salt stress and/or general starvation.

10. A transformation vector, comprising a polynucleotide according to claim 1-9.

11. An eukaryotic cell, transformed with a transformation vector according to claim 10.

12. A transgenic animal, transformed with a transformation vector according to claim 11.

13. A transgenic plant, transformed with a transformation vector according to claim 11.

14. A method for facilitating cap independent translation of mRNA in an eukaryotic cell, by incorporating a polynucleotide according to claim 1-9 before a coding sequence.

15. A method for facilitating stress induced translation of mRNA in an eukaryotic cell, by incorporating a polynucleotide according to claim 1-9 before a coding sequence.


SEQUENCE LISTING (29 sequences)

SEQ ID NO: 1 - RNA, 12 nt, Artificial Sequence
  Description of Artificial Sequence: inhibitor oligonucleotide
uuguuacgacuu

SEQ ID NO: 2 - RNA, 11 nt, Arabidopsis thaliana
cuucuucuucu

SEQ ID NO: 3 - RNA, 11 nt, Artificial Sequence
  Description of Artificial Sequence: IRES
cuucuycuucy

SEQ ID NO: 4 - DNA, 22 bp, Artificial Sequence
  Description of Artificial Sequence: gene RPS18A
tggtggcgcctccagagtctgg

SEQ ID NO: 5 - DNA, 22 bp, Artificial Sequence
  Description of Artificial Sequence: gene RPS18B
ttctcaggcatctcttatcttc

SEQ ID NO: 6 - DNA, 20 bp, Artificial Sequence
  Description of Artificial Sequence: gene RPS18C
acggcttcttcttctcacaa

SEQ ID NO: 7 - DNA, 22 bp, Artificial Sequence
  Description of Artificial Sequence: common primer
gtcatgaggttatcaatctcag

SEQ ID NO: 8 - DNA, 23 bp, Artificial Sequence
  Description of Artificial Sequence: primer
  misc_feature (10)..(15): BamHI restriction site
cctcttttgggatcctcactctc

SEQ ID NO: 9 - DNA, 25 bp, Artificial Sequence
  Description of Artificial Sequence: primer
  misc_feature (8)..(13): NcoI restriction site
ctaattaccatggtgattagcagag

SEQ ID NO: 10 - DNA, 20 bp, Artificial Sequence
  Description of Artificial Sequence: primer
  misc_feature (11)..(16): NcoI restriction site
actattctagccatggagaa

SEQ ID NO: 11 - DNA, 20 bp, Artificial Sequence
  Description of Artificial Sequence: primer
ccatacggaattccggatga

SEQ ID NO: 12 - DNA, 35 bp, Artificial Sequence
  Description of Artificial Sequence: oligo
cgatctggaattaaaatgtctaaaaaaatcactgg

SEQ ID NO: 13 - DNA, 33 bp, Artificial Sequence
  Description of Artificial Sequence: mutagenic oligonucleotide #110
gtttattgcttgaagtgctgaacttcttctcac

SEQ ID NO: 14 - DNA, 37 bp, Artificial Sequence
  Description of Artificial Sequence: mutagenic oligonucleotide #111
gcttgaagacggcttgaggaaggcacaaacctcatct

SEQ ID NO: 15 - DNA, 25 bp, Artificial Sequence
  Description of Artificial Sequence: mutagenic oligonucleotide #112
acggcttcttgaactcacaaacctc

SEQ ID NO: 16 - RNA, 18 nt, Artificial Sequence
  Description of Artificial Sequence: primer oligo #1
acgacuucuccuuccucu

SEQ ID NO: 17 - RNA, 12 nt, Artificial Sequence
  Description of Artificial Sequence: primer oligo #2
ucuccuuccucu

SEQ ID NO: 18 - RNA, 18 nt, Artificial Sequence
  Description of Artificial Sequence: primer oligo #3
acgacuucuggaaggucu

SEQ ID NO: 19 - RNA, 12 nt, Artificial Sequence
  Description of Artificial Sequence: random oligo
gaucgaucgauc

SEQ ID NO: 20 - RNA, 7 nt, Artificial Sequence
  Description of Artificial Sequence: activator sequence
acgacuu

SEQ ID NO: 21 - RNA, 8 nt, Artificial Sequence
  Description of Artificial Sequence: effector sequence
cuucuucu

SEQ ID NO: 22 - RNA, 107 nt, Unknown
  Predicted structure of RPS18C leader
  stem_loop (5)..(15)
  stem_loop (22)..(38)
  stem_loop (40)..(54)
  misc_feature (44)..(58): complementary sequences to 18S rRNA
  stem_loop (65)..(70): stem portion of stem-loop paired with residues 101-106
  misc_feature (40)..(44)
  stem_loop (74)..(99)
  stem_loop (82)..(92): loop portion of stem loop
  stem_loop (101)..(106): stem portion of stem loop paired with residues 65-70
  stem_loop (7)..(13): loop portion of stem loop
  stem_loop (27)..(33): loop portion of stem loop
  stem_loop (44)..(50): loop portion of stem loop
cuuuuguguucuucacucuccagcgaucguuuauugcuugaagacggcuucuucuucucacaaccucaucucugcuaaucaaaaugucucugguugcaaaugaggag

SEQ ID NO: 23 - RNA, 107 nt, Unknown
  Alternative predicted structure of RPS18C leader
  misc_feature (44)..(58): complementary sequences to 18S rRNA
  stem_loop (9)..(14): stem portion of stem loop paired with residues 39-44
  stem_loop (22)..(38)
  stem_loop (27)..(33): loop portion of stem loop
  stem_loop (39)..(44): stem portion of stem loop paried with residues 9-14
  stem_loop (59)..(64): stem portion of stem loop paried with residues 3-8
  stem_loop (65)..(70): stem portion of stem loop paried with residues 101-106
  stem_loop (3)..(8): stem portion of stem loop paried with residues 59-64
  stem_loop (101)..(106): stem portion of stem loop paried with residues 65-70
  stem_loop (74)..(99)
  stem_loop (82)..(92): loop portion of stem loop
cuuuugugucuucacucuccagcgaucguuuauugcuugaagacggcuucuucuucucacaaaccucaucucugcuaaucaaaaugucucugguugcaaaugaggcg

SEQ ID NO: 24 - RNA, 86 nt, Unknown
  RPS18C leader sequence
  misc_feature (44)..(58): complementary sequence to the 18s rRNA
cuuuuguguucuucacucuccagcgaucguuauugcuugaagacggcuucuucuucucacaaaccucaucucugcuaaucaaaaug

SEQ ID NO: 25 - RNA, 80 nt, Unknown
  3′ end of 18s rRNA from positions 1645 to 1803
  Unsure (13)..(24): corresponds to positions 1657 and 1747 in full length sequence-helix 49
  stem_loop (3)..(10): stem portion of stem loop paired with residues 27-34
  stem_loop (27)..(34): stem portion of stem loop paired with residues 3-10
  stem_loop (14)..(23)
  stem_loop (16)..(21): loop of stem loop
  stem_loop (49)..(72)
  stem_loop (58)..(63): loop portion of stem loop
cgcuccuaccgauugguucgccgagaggaaggagaagucguaacaagguuuccguaggugaaccugcggaaggaucauug

SEQ ID NO: 26 - RNA, 15 nt, Unknown
  RPS18C - 18s rRNA complementary region
acggcuucuucuucu

SEQ ID NO: 27 - DNA, 15 bp, Artificial Sequence
  Description of Artificial Sequence: gene RSP18C stem loop sequence
acggcttcttcttct

SEQ ID NO: 28 - DNA, 15 bp, Artificial Sequence
  Description of Artificial Sequence: gene RSP18C stem loop sequence mutated
tgctgaacttcttct

SEQ ID NO: 29 - DNA, 15 bp, Artificial Sequence
  Description of Artificial Sequence: gene RSP18C stem loop sequence mutated
acggcttcttgaact